(19) United States
(12) Patent Application Publication
(10) Pub. No.: US 2010/0056922 A1
(43) Pub. Date: Mar. 4, 2010

(54) Method and diagnostic ultrasound apparatus for determining the condition of a person's artery or arteries

(76) Inventor: Thierry Florent, Napa, CA (US)

Correspondence Address: Zachary T. Wobensmith, III, 7746 101st Court, Vero Beach, FL, 32967-2871, US

(21) Appl. No.: 12/231,273

(22) Filed: Sep. 2, 2008

(51) Int. Cl. A61B 8/06

(52) U.S. Cl. 600/454

(57) ABSTRACT

A method and diagnostic apparatus for determining the condition of a patient's artery or arteries by the use of an ultrasound imaging system which operates in the triplex mode, with a B-mode image of a selected artery location, an A-mode perpendicular to the plane of the artery, at the selected location and a pulsed doppler, at the selected location at an angle to the plane of the artery, with the signals combined, the artery physically or chemical stimulated, the percent dilation of the artery is determined after stimulation, and therefore the condition of the artery is obtained.

### BACKGROUND OF THE INVENTION

1. Field of the Invention

This invention relates to a method and diagnostic ultrasound apparatus for determining the condition of a person's artery or arteries, by using an ultrasound system which shares the PRF of its modes to provide a triplex mode, with a B-mode image displaying a selected artery location, an A-mode cursor perpendicular to the artery, and a pulsed doppler cursor at a 60-degree angle to the artery.

2. Description of the Prior Art

Cardiovascular disease causes more than 925,000 deaths annually in the United States alone. It has an economic burden of 128 billion dollars. This disease is highly prevalent and its progression may be preventable if detected and treated early in its onset.

Cardiovascular disease begins to develop shortly after birth. Autopsies have shown that 50 to 75% of young men have coronary atherosclerosis with 5 to 10% having a high grade of stenosis. More than 75% of elderly individuals will have a high-grade stenosis.

There are numerous causes of cardiovascular disease such as elevated serum Lipids, hypertension, diabetes mellitus, smoking, elevated homocysteine levels, decreased estrogen, renal failure and many other causes. Effective treatment of these disorders has been shown to reduce morbidity and mortality by 5 to 40%. The treatment of large populations as early as possible reduces the incident of cardiovascular death, however successful treatment of an individual is much less certain. At this time, we cannot tell if we have treated that individual for the right problems, or treated him or her sufficiently to ward off the progression of cardiovascular disease. The degree of control of cardiovascular disease in an individual is currently very difficult to measure.

The wall of an artery is made up of three layers. The innermost layer touching the blood is called the intima. The next layer consists mostly of smooth muscle cells and is the media. The outermost layer is comprised of fibrous tissue, and is the adventitia. The intima has several parts. There is single layer of cells in contact with the blood called endothelial cells. Behind these cells is a thin layer of connective tissue (strands of fiber), possibly a few smooth muscle cells, and the basement membrane, which separates the intima from the media.

Artherosclerosis develops between the endothelium and the basement membrane. Cholesterol and white cells from the blood stream penetrate the endothelial barrier, and then form plaque between the endothelium and the basement membrane. When these plaque deposits become large, they protrude into the artery and eventually close off the artery.

Healthy endothelium is a very dynamic chemical factory. It can produce many substances, some of which keep plaque from forming. If the endothelium is depressed it reduces the production of and or stops making many of its beneficial chemicals, and may produce substance that can promote plaque formation. It is known that smoking, high blood pressure, etc. depresses endothelium function.

Measuring the health of the endothelium gives us a measurement of the artherosclerosis process and the effectiveness of treatment. Healthy endothelium produces nitric oxide, depressed endothelium does not. Nitric oxide helps control the diameter of an artery. The more nitric oxide produced the larger the diameter of the artery. There are a number of factors that will cause healthy endothelium to produce more nitric oxide. One stimulus is to increase the blood flow in the artery, such as when a muscle is being used. The increased blood flow in the artery increases shear forces on the endothelium as it flows down the artery. The endothelium sense the higher flow, then secretes more nitric oxide and dilates the artery. When the flow returns to normal, the cells reduce nitric oxide secretion, and the artery decreases in diameter.

If you place a blood pressure cuff around the forearm of an individual, and inflate it to greater than arterial pressure, blood flow will cease in the arm down stream from the cuff. The arm and hand below the cuff will develop an oxygen deficiency. When the cuff is released, the blood flow to the arm and hand is very high. When the oxygen is replaced, the blood flow returns to normal.

The blood to the forearm is provided through the brachial artery on the inside of the upper arm, which is the artery of choice for monitoring by cardiologists. The diameter of the brachial artery can be measured using diagnostic ultrasound. When the blood flow is increased through the brachial artery, it dilates because of increased nitric oxide secretion, and this dilation is measured by using diagnostic ultrasound. If the endothelium is healthy, the artery can be made to dilate 10 to 20%. If the endothelium is depressed, the artery will dilate only 1 or 2%. By monitoring the artery dilation, we now have a measuring tool to determine the degree of success in treating artherosclerosis.

Various treatment regimes for cardiovascular disease are used, many of which use various pharmaceuticals to lower cholesterol, to decrease the formation of plaque, and to increase blood flow in the artery being monitored. One of the problems with many of these pharmaceuticals is that it is difficult to measure the treatment progress or lack thereof, and visible results may take many months to be recognized.

Thus one of the goals of cardiology is to be able to rapidly measure the progress of any given treatment regime. No one has been successful in terms of finding a technique to hasten measuring the results of treatment. Brachial artery studies have been conducted which look at the response of the endothelium to various drug regimes. It is common to measure the nitric oxide production of endothelium, and if it is increasing, you know that the outcome is progressing in a favorable fashion. How you improve the health of the endothelium so that the endothelium will produce more nitric oxide is important, since producing more nitric oxide will slow down, stabilize or actually reverse the artherosclerotic process.

If you can measure nitric oxide, and if it goes up, you know that the artherosclerosis is abating, or if it goes down another treatment regime may be required. The health of the endothelium can be measured by checking arterial stiffness, such as checking reflected pressure waves in the arterial system. You can measure arterial stiffness by putting catheters inside arteries, or make a measurement by putting on an external device that will measure the pressure waves, and the reflected pressure waves in the arteries.

The problem with these techniques is that you can only measure artherosclerosis after it has reached a very advanced state. You cannot deduce it early on and the above measuring techniques are insensitive to changes in arterial dilation. That is, it still takes six months to a year to see any kind of useful change in the patient's condition following the institution of the medical regimen.

Imaging the brachial artery with an ultrasound system and obtaining the diameter of the artery is not a difficult process. The brachial artery is usually between 2.5 mm and 7 mm in diameter or ⅛ inch in size. Measurements can be made to an accuracy of 0.15-2 mm in the best case. This would give us an error of between 2 and 6%. Thus if we measured the dilatation of a brachial artery and found it to be 8% it is actually somewhere between 6 and 10% best case. This does not give us sufficient accuracy to evaluate an individual's treatment progress.

A diagnostic apparatus was developed using ultrasound and a wall-tracking device that included A-mode (radio frequency) waves. This system only measured the diameter of an artery to within 0.15 mm. The unique feature of a wall tracking system is that it can lock onto the wall of the artery and track movement of the wall to within 0.001 mm. We do not need to know the absolute diameter of an artery but how much the diameter changes during the study. Using a wall tracking system, we now can measure changes in artery diameter to within 1%, and thus accurately determine the percent dilation of the artery, and the progress or lack of progress of a treatment regime.

### SUMMARY OF THE INVENTION

This invention relates to a diagnostic ultrasound apparatus which uses sharing of the PRF of all the modes to provide a triplex mode of B-mode imaging to locate an artery to be measured, an A-mode cursor perpendicular to the artery, and a pulsed doppler cursor at a 60-degree angle to the artery to measure the arterial diameter before and after chemical, or physical stimulation, and determine the percentage of arterial dilation of the artery and the progress or lack of progress of a treatment regime.

The principal object of the invention is to provide a method and diagnostic ultrasound apparatus for determining the condition of a person's artery or arteries by using an ultrasound apparatus in the PRF shared triplex mode, with a B-mode for artery location, an A-mode cursor perpendicular to the artery, and a pulsed Doppler cursor at a 60-degree angle to the artery, which combination allows us to measure the percent dilation of the artery upon chemical or physical stimulation.

A further object of the invention is to provide a method and apparatus of the character aforesaid which provides a high degree of accuracy.

A further object of the invention is to provide a method and apparatus of the character aforesaid which is simple to use.

A further object of the invention is to provide a method and apparatus of the character aforesaid which uses conventional ultrasound apparatus that has been modified to share the PRF of all modes so as to operate in the triplex mode.

A further object of the invention is to provide a method and apparatus of the character aforesaid which is simple to construct but sturdy and reliable in operation.

Other objects and advantageous features of the invention will be apparent from the description and claims.

### DESCRIPTION OF THE DRAWINGS

The mature and characteristic features of the invention will be more readily understood from the following description taken in connection with the accompanying drawings forming part hereof in which:

FIG. 1 is a plan view of the apparatus of the invention;

FIG. 2 is a perspective view of a typical brachial artery;

FIG. 3 is a block diagram of the apparatus of the invention for artery diameter and blood flow;

FIG. 4 is a typical display generated by the apparatus of the invention of an artery being measured;

FIG. 5 is two graphs illustrating blood flow and artery diameter;

FIG. 6 is a flow chart of the patient's wall tracker flow;

FIG. 7 is a flow chart of the patient's process diameter section of the wall tracker.

FIG. 8 is a flow chart of the patient's heartbeat level distension section of the wall tracker, and

FIG. 9 is a flow chart of the patient's Doppler processing.

It should, of course, be understood that the description and drawings herein are merely illustrative, and that various modifications and changes can be made in the method and apparatus disclosed without departing from the spirit of the invention.

Like numerals refer to like parts throughout the several views.

### DESCRIPTION OF THE PREFERRED EMBODIMENT

When referring to the preferred embodiments, certain terminology will be utilized for the sake of clarity. Use of such terminology is intended to encompass not only the described embodiment, but also technical equivalents, which operate and function in substantially the same way to bring about the same result.

Referring now more particularly to FIG. 2 of the drawings, a perspective view of a typical brachial artery 10 is therein illustrated. The wall of artery 10 is made up of three layers, an innermost layer 11, which is the intima, a center layer 12 which consists mostly of smooth muscle cell, and is the media, and an outermost layer 14 of fibrous tissue, which is the adventita.

The intima 11 has a single layer 15, of cells in contact with the blood (not shown) known as the endothelium, a thin layer 16 of connective tissue (fiber strands) and possibly a few muscle cells, and the basement membrane 20, which separates the intima from the media.

Plaque deposits (not shown) develop in the intima between the endothelium layer 15 and the basement membrane 20.

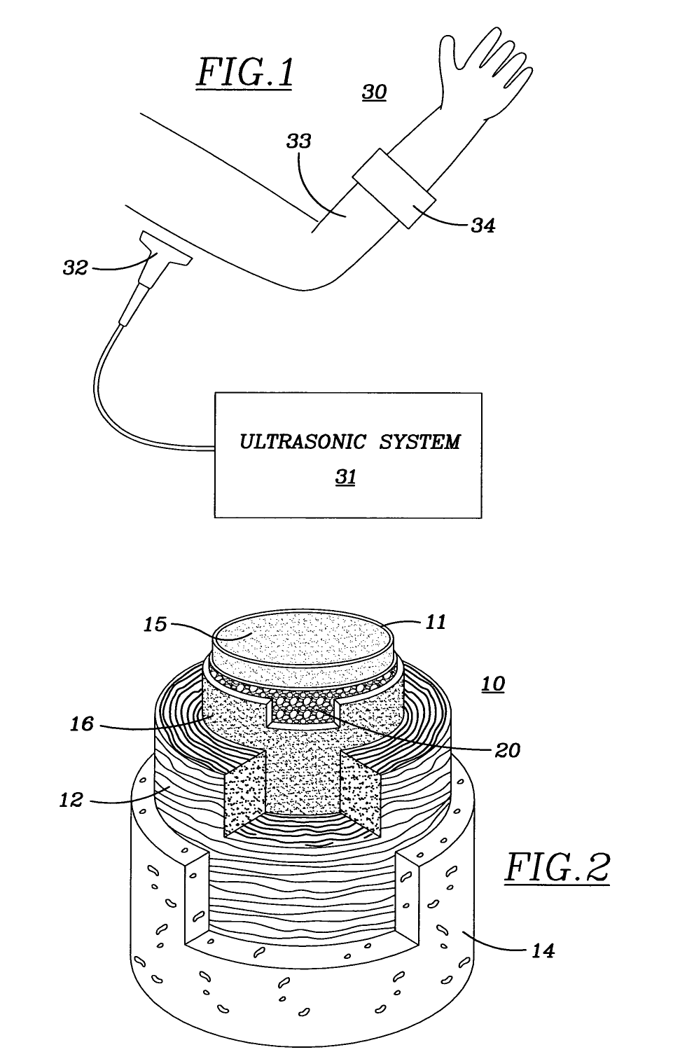

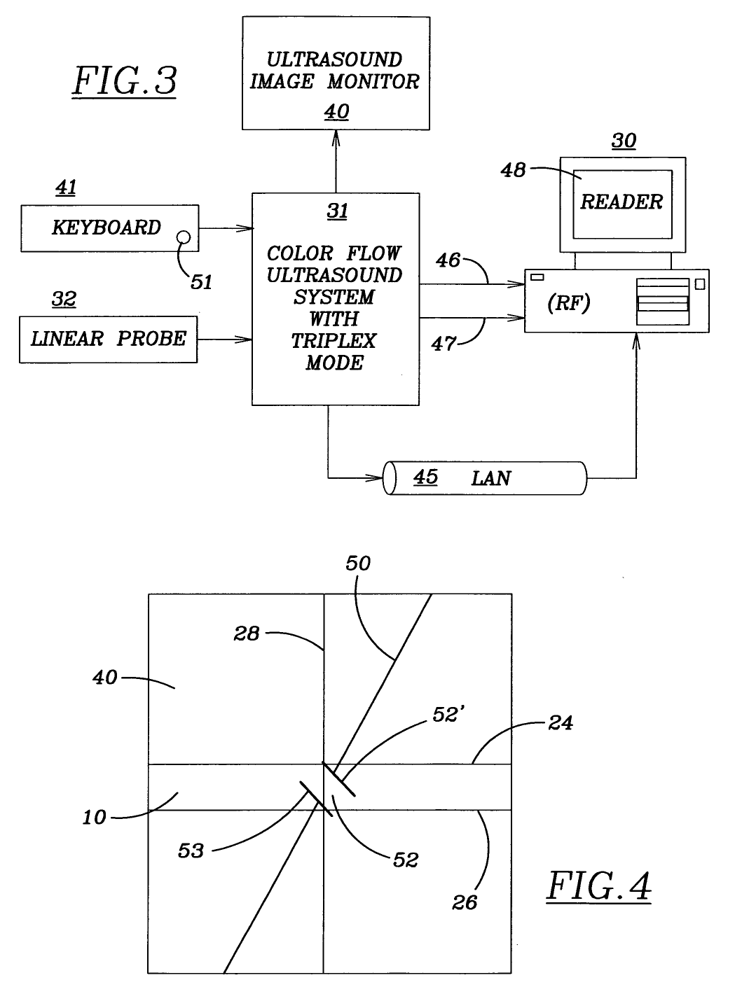

Referring additionally to FIGS. 1, 3 a typical human arm 30 is therein illustrated, with an ultrasound system 31 to be described, which includes a standard well known transducer 32 extending therefrom, and adjacent to the forearm 33 of arm 30. The drive of the transducer 32 is done by well-known programming, and the electronics of the ultrasound system 31.

In one embodiment, the standard transducer 32 includes an array of elements of 128 crystals, with the crystals that are fired determined by the control electronics in the ultrasound system 31. If operating in the A-mode, one of those crystals is selected for transmission. If operating in a Doppler mode, you use just one crystal on transmit. Note that a number of crystals are used to listen to the reflected wave. Thus you use a single crystal to transmit and multiple crystals to receive. It will thus be appreciated that in the subject system a single transducer is used that is first operated in the A-mode and then in the Doppler mode. An inflatable cuff 34 of well known type is illustrated on forearm 33, for controlling blood flow through arm 30, to be described. The Ultrasound apparatus 31 can be any desired system, with a preferred system being the Tetrad 2300 Color flow system, available from W. L. Gore & Associates, Inc., Denver, Colo., which includes a beamformer which has been modified to share the pulse repetition frequency (PRF) to provide a triplex mode.

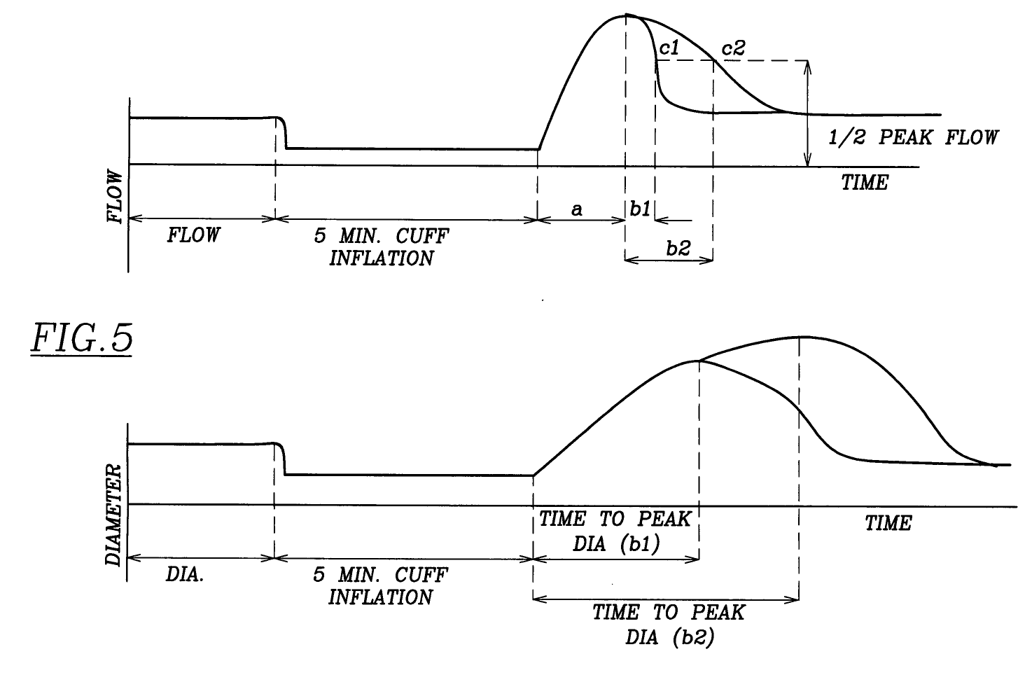

Referring additionally to FIGS. 4, 5 an ultrasound display 40 is illustrated, which includes an artery 10, preferably the brachial artery of arm 30, which has an anterior wall 24 and a posterior wall 26. An A-mode cursor 28 is superimposed on the display 40, perpendicular to the longitudinal axis of artery 10, which is at the position of acquisition of an A-mode signal.

A Doppler cursor 50 is shown on display 40, and is disposed at an angle with respect to A-mode cursor 28, preferably 60 degrees. A Doppler pulse gate 52, of the Doppler cursor 50 is positioned inside artery 10 by a track ball 51 as shown in FIG. 3, represented by lines 52 and 53, and as described below. The track ball 51 positions the A-mode cursor for left to right (horizontal) motion in response to left to right track ball input. The Doppler gate 52 is positioned along the Doppler cursor 50, between the anterior wall 24 and posterior wall 26 of artery 10 by using the up and down motion (vertical) of the track ball 51.

Measuring endothelium health using the brachial artery dilatation method consists of a few steps. The person whose artery is to be measured must be lying flat on an examining table in a relaxed position. The cuff 34 is placed on the arm 30, which must extend outwardly, and be fixed in place so it cannot move during the study. The transducer 32 is placed adjacent the arm 30, and the system 31 activated. The blood pressure cuff 34 is inflated to a pressure of 20 mmHg greater than systolic blood pressure. Approximately 5 minutes later the cuff 34 is released, and the blood flow and the artery diameter are measured by use of the transducer 32 for 3 to 5 minutes to be described.

The blood flow measured in the brachial artery 10 is usually 10 to 15 cm/sec. When the blood pressure cuff 34 is inflated on the forearm 33, the blood flow in the brachial artery 10 decreases. Upon release of the cuff 34, the blood flow in the brachial artery 10 rises sharply to a maximum value. The time from cuff 34 release until maximal blood flow “a” is approximately 3 seconds. Point “c” is the point at which blood flow has fallen to half of its maximal value. The time from maximal flow to one-half maximal flow is time “b”. If the blood pressure cuff 34 is applied for 2 minutes, time “b” is very short, if the cuff 34 is applied for 10 minutes, time “b” is much longer.

In FIG. 5, the bottom graph shows what happens to the diameter of the brachial artery 10 during the test. The normal brachial artery 10 diameter decreases when the cuff 34 is applied because brachial artery blood flow is decreased, and there is less nitric oxide production. Upon release of the cuff 34, the higher blood flow now stimulates increased nitric oxide production and dilation of the artery 10, but this is a slower process than the increase in blood flow. The endothelial cells 15 have to become aware of the higher blood flow and then generate nitric oxide. The nitric oxide diffuses from the cells 15 through the intima 11, and into the smooth muscle cells of the media 12. These muscle cells must relax and allow the artery 10 to dilate, which process is slow. The time to maximum artery 10 dilation is about 40 seconds in young healthy individuals. It may take 2 to 3 minutes to reach maximum artery dilation in people with depressed endothelium.

The maximum diameter reached by the brachial artery 10 depends upon how strongly the nitric oxide machinery was turned on. This stimulus strength is measured by time “b” seen in the blood flow diagram. The longer it takes to reach point “c” the longer the time “b”, and the more the brachial artery 10 dilates in a normal individual.

The triplex mode color flow ultrasound system 31, as illustrated in detail in FIG. 3 includes a display monitor 40, a keyboard 41 for controlling the ultrasound system, and a transducer 32 for collecting patient data. By means of a local area network connection (LAN) 45, digitized RF data is sent to a reader 44 for storage and analysis and is displayed on video display 48. Doppler I and Q signals 46 from the system 31, are inputted to the reader 44 audio input for Doppler spectrum analysis and display. SVHS video from system 31 is inputted to the reader's frame grabber input for storage and display on the video display 48.

Referring to FIG. 4, the B-mode display is used to place the A-mode cursor 28 over the center of the artery 10 in the longitudinal plane, with the anterior wall 24 and posterior wall 26 displayed as shown.

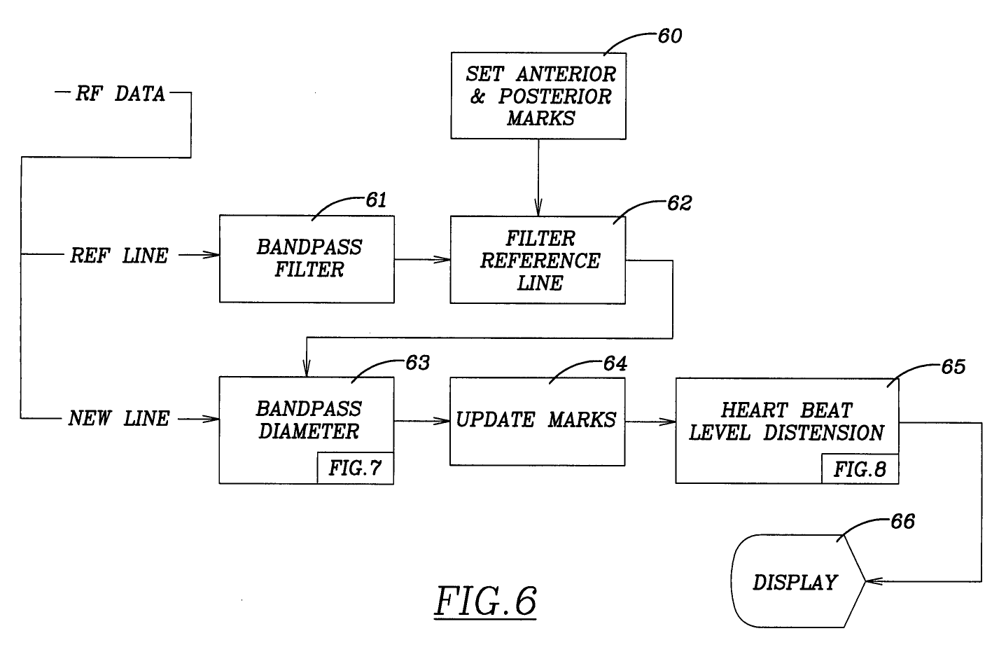

Referring to FIG. 6 of the flow charts, the digitized RF data is first band pass filtered 61 about the center frequency of the transducer 32. The first A-line of RF data is stored as the reference line 62. Markers 60 are then placed on the anterior and posterior walls 24 and 26, on the inside of the artery 10 reference line.

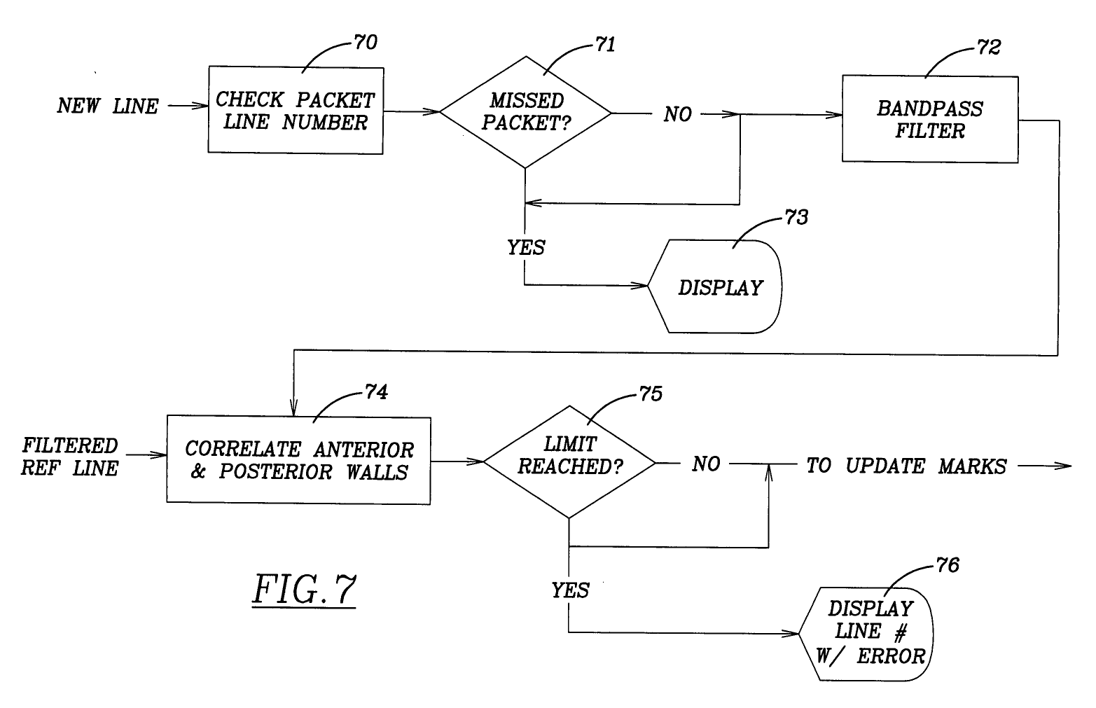

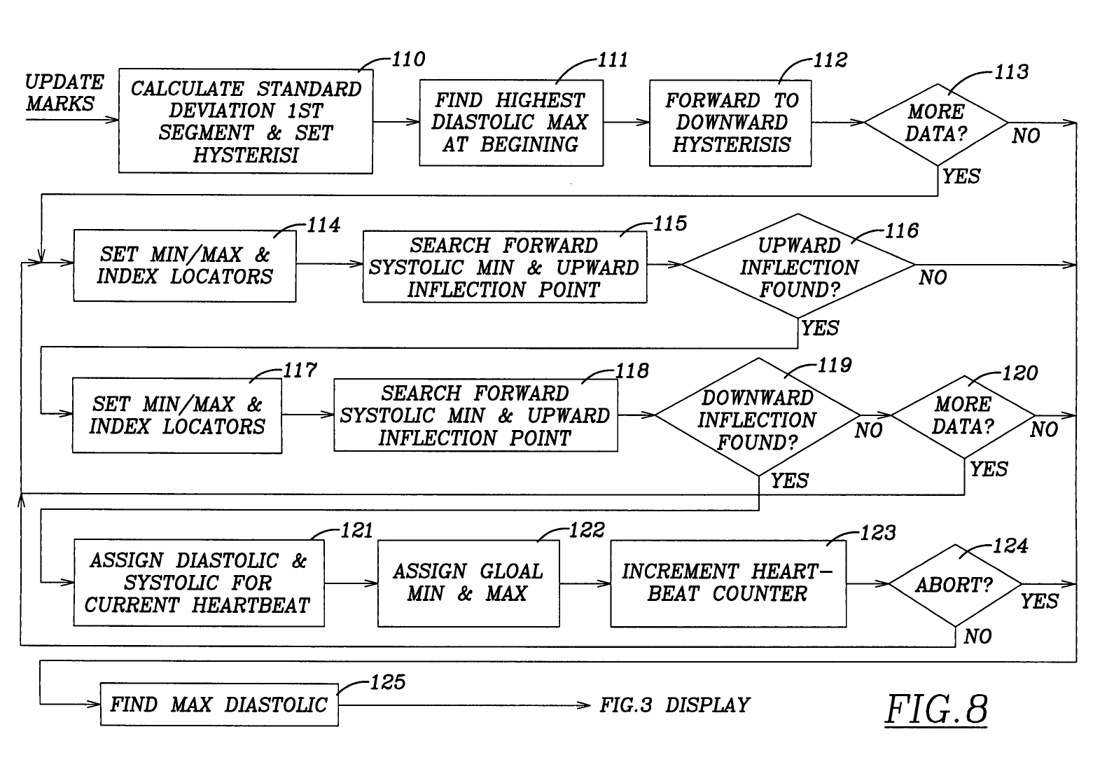

In FIG. 7 the subsequent new lines are checked for missing packets 70. If a missed packet is detected 71, the missing line number is displayed 73. The new line is band pass filtered 72, about the center frequency of the transducer 32. The new lines are then compared to the reference line by correlation 74, to measure the anterior and posterior walls movement in digitizer sample clock cycles. The new position is checked for errors by comparing and reporting errors to the display 76.

Referring again to FIG. 6, the new position is used to update the markers 64. Once all of the wall position data is stored, the diastolic minimum and maximum pressures are identified by the Heart Beat Level Distension 65, and the percent dilation is calculated by the following formula:

Percent dilatation=100×(diastolic maximum−diastolic minimum)/diastolic minimum.

The percent dilatation result is sent to the display 66.

Referring to FIG. 8, the updated marks which contain all wall position information, is used to calculate the standard deviation 110 of the distension data to check that the data is within limits and to adapt to changing amount of dilation by setting a hysteresis value.

The highest diastolic maximum at the beginning of the study is located 111, and from there moved forward to the downward hysteresis point 112. If there is more data 113, the minimum and maximum index pointers are set 114. Searching forward 115, the systolic minimum and upward inflection point is located. If the upward inflection point is found 116, the minimum and maximum index pointers are set again 117, from there search forward for the diastolic maximum 118, until the downward inflection point is found 119. If found, the diastolic and systolic values for the current heartbeat are assigned 121, and the global variables for the minimum and maximum are updated 122. The heartbeat counter is incremented 123. If at any time there is no more data to process 113 and 120, or an abort occurs 124, flow continues to find the maximum diastolic value 125.

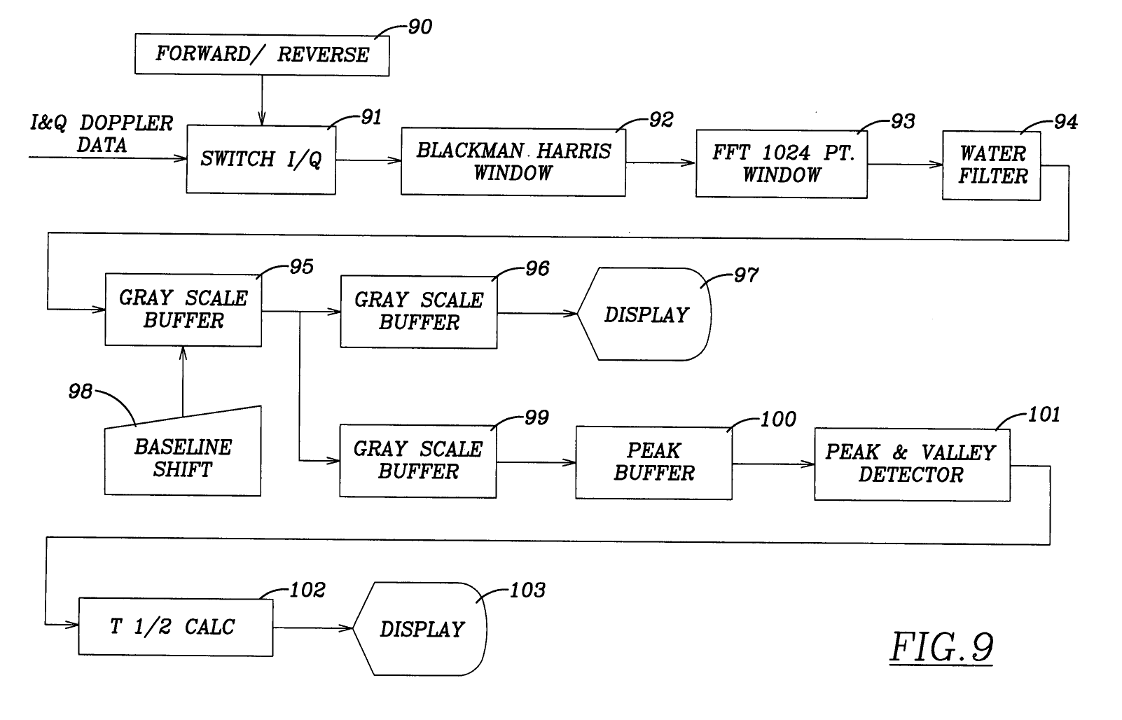

Referring to FIG. 9, I and Q Doppler data from disk is inputted to switch 91, with the user having the option to reverse spectrum direction 90.

The I and Q Doppler data is windowed 92, by a Blackman Harris minimum 4 term. A 1024 point fast Fourier transformer, FFT 93, returns Doppler spectrum data that is low pass filtered 94 to remove wall movement artifacts.

The processed spectrum is stored in a first Gray scale buffer 95, with the user having the option to shift baseline 98 to prevent wrap around of spectrum peaks. The baseline-shifted spectrum is stored in a second Gray scale buffer 96 and displayed 97. The shifted spectrum has the peak envelope detected 99 and stored 100. The peak and valley detector 101 locates the diastolic and systolic points. Using the diastolic points to mark the beginning of one heartbeat and the start of the next the T ½ calc 102, calculates the time required for the flow to lower to ½ of its maximum value, and is displayed 103.

In one embodiment, the dilation percent is corrected by the diameter of the artery. The larger the artery, the less percent dilation you would expect to get in a normal individual.

As to blood flow, blood flow is determined by the area under the curve of the blood flow. In other words, you measure how high the velocity goes and for how long over normal blood flow. This measurement corrects for the expected normal percent dilation.

In other words, if a patient has a very high blood flow over a long period of time, you would expect a normal percent dilation to be somewhere around 15%. If the patient's artery only dilates 5%, then you know that their endothelium is very thick. You would then integrate the flow over time and that becomes a number, which you multiply times a constant.

What this means is that a normal dilation for somebody with high blood flow might be 15%, but with somebody with low blood flow because of whatever reason, they might be perfectly healthy at 10%.

It will thus be seen that a Method and Ultrasonic Diagnostic Apparatus have been described with which the objects of the invention are achieved.

1. A method for measuring the condition of a patient's artery comprising the steps of:
measuring the diameter and blood flow from an artery by an ultrasound transducer,
stimulating said artery to produce nitric oxide;
measuring the diameter and blood flow from said artery using the same ultrasound transducer, and;
deducing the condition using the two measured quantities.

2. The method of claim 1, wherein the measurement includes detecting percent dilation of the artery.

3. The method of claim 2, wherein the percent dilation occurs after arterial stimulation.

4. The method of claim 3, wherein the artery is in an extremity, and wherein the stimulation includes inflating a blood pressure cuff around the extremity.

5. The method of claim 3, wherein the artery diameter is correlated with blood flow at the point at which the artery diameter is measured.

6. The method of claim 1, wherein the measuring step is done using ultrasonic apparatus operating specifically in the A-mode and pulsed Doppler modes in which energy is projected into the artery from a single transducer.

7. The method of claim 6, wherein the position of the artery is determined by using an ultrasound B-mode using the same transducer used in the A-mode and Doppler modes.

8. The method of claim 6, wherein the measuring step includes using the A-mode for arterial diameter measurements and the Doppler mode for measuring blood flow.

9. The method of claim 8, wherein the A-mode energy is transmitted normal to the longitudinal axis of the artery and wherein the Doppler mode is transmitted at an angle to the longitudinal axis of the artery.

10. The method of claim 9, wherein the transducer includes an array of elements and wherein the angle of the transmitted signal with respect to the longitudinal axis of the artery is determined by the phasing of the elements.

11. A method for determining the condition of an artery comprising the steps of:
causing a stimulus that produces of nitric oxide in an artery, and;
measuring the diameter and blood flow through the artery from a single transducer when the artery is at rest and after stimulation, thus to generate a measurement relating to percent dilation of the artery in response to the stimulus.

12. An ultrasound imaging apparatus for determining the condition of a patient's artery or arteries by measuring diameter and blood flow in the same location which comprises;
an ultrasound system,
said system including a transducer to image the patient's artery,
said system operating in a B-mode to locate a selected location of said artery, an A-mode projecting an RF signal to said selected location perpendicular to said artery, and a pulsed doppler projecting a signal to said selected location at an angle to the plane of the artery to generate I and Q doppler signals,
a local area network to transmit the received RF data to a reader for storage and analysis, means to transmit said I and Q doppler signals to said reader for storage and analysis, whereby compilation of said RF data and said signals provides the percent dilation of a patient's artery.

13. Apparatus as defined in claim 12 in which, said pulsed doppler signals are at a 60 degree angle to said artery.

14. Apparatus as defined in claim 12 in which; said ultrasound system is a Color Flow Ultrasound System operating in a Triplex mode.

15. Apparatus as defined in claim 12 in which said ultrasound system includes an ultrasound image monitor, and a keyboard.

16. Apparatus as defined in claim 12 in which: said transducer has 128 crystals.
